(19) United States
(12) Patent Application Publication
(10) Pub. No.: US 2026/0354690 A1
(43) Pub. Date: Nov. 12, 2026

(54) IDENTIFYING RESPONDER ID USING PHOTOACOUSTIC IMAGING

(71) Applicant: Medtronic Ireland Manufacturing Unlimited Company, Dublin 2 (IE)

(72) Inventors: Binit Panda, Maple Grove, MN (US); Jingkuang Chen, Santa Rosa, CA (US)

(73) Assignee: Medtronic Ireland Manufacturing Unlimited Company, Dublin 2 (IE)

(21) Appl. No.: 19/132,664

(22) PCT Filed: Nov. 16, 2023
(86) PCT No.: PCT/EP2023/082090

§ 371 Date: May 23, 2025

Related U.S. Application Data
(60) Provisional application No. 63/427,624, filed on Nov. 23, 2022.

(51) Int. Cl. A61B 5/00; A61B 5/021; A61B 18/00; A61B 18/02; A61B 18/14

(52) U.S. Cl. CPC A61B 5/0095; A61B 5/02125; A61B 5/4848; A61B 18/1492; A61B 2018/00577; A61B 2018/0212

(57) ABSTRACT

A system and method for performing a diagnostic procedure, the method including causing, a light source to deliver a first series and a second series of laser pulses to a first location and a second location along a wall of a blood vessel The method includes receiving a first signal indicative of detected ultrasonic waves emitted in response to the first series of laser pulses and receiving a second signal indicative of detected ultrasonic waves emitted in response to the second series of laser pulses, determining a difference in time between emission of maximum ultrasonic waves in response to the first series of laser pulses and emission of a maximum ultrasonic waves in response to the second series of laser pulses, calculating a pulse wave velocity within the blood vessel based on a distance between the first location and the second location and the determined difference in time.

This application claims the benefit of U.S. Provisional Patent Application Ser. No. 63/427,624, filed Nov. 23, 2022, the entire content of which is incorporated herein by reference.

### TECHNICAL FIELD

This disclosure relates generally to neuromodulation and associated systems and methods. In particular, the disclosure is directed to diagnostic methods and systems for pre-procedurally determining potential efficacy of neuromodulation therapy for a particular patient and peri-procedurally assessing the efficacy of a neuromodulation therapy.

### BACKGROUND

Catheters have been proposed for use with various medical procedures. For example, a catheter can be configured to deliver neuromodulation (e.g., denervation) therapy to a target tissue site to modify the activity of nerves at or near the target tissue site. The nerves can be, for example, sympathetic or parasympathetic nerves. The sympathetic nervous system (SNS) is a primarily involuntary bodily control system typically associated with stress responses. Chronic over-activation of the SNS is a maladaptive response that can drive the progression of many disease states. For example, excessive activation of the renal SNS has been identified experimentally and in humans as a likely contributor to the complex pathophysiology of arrhythmias, hypertension, states of volume overload (e.g., heart failure), and progressive renal disease.

Percutaneous renal denervation is a minimally invasive procedure that can be used to treat hypertension and other diseases caused by over-activation of the SNS. During a renal denervation procedure, a clinician delivers stimuli or energy, such as radiofrequency, ultrasound, cooling, or other energy to a treatment site to reduce activity of nerves surrounding a blood vessel. The stimuli or energy delivered to the treatment site may provide various therapeutic effects through alteration of sympathetic nerve activity.

### SUMMARY

One aspect of the disclosure is directed to a system for performing a diagnostic procedure. The system includes a medical device configured for placement within a blood vessel and including a light source and an ultrasound transducer disposed on a distal portion of the medical device. The system also includes a computing device including a processor and a memory storing instructions, which when executed by the processor, cause the computing device to: cause the light source to deliver a first series of laser pulses to a first location along a wall of a blood vessel; cause the light source to deliver a second series of laser pulses to a second location along a wall of the blood vessel; receive a signal indicative of ultrasonic waves emitted in response to the first series of laser pulses from the ultrasound transducer; receive a second signal indicative of ultrasonic waves emitted in response to the second series of laser pulses from the ultrasound transducer; determine a difference in time between emission of maximum amplitude of ultrasonic waves in response to the first series of laser pulses and emission of a maximum amplitude of ultrasonic waves in response to the second series of laser pulses; and calculate a pulse wave velocity within the blood vessel based on the calculated a distance between the first location and the second location and the determined difference in time, where a pulse wave velocity in excess of a threshold is predictive of a response to therapy. Other embodiments of this aspect include corresponding computer systems, apparatus, and computer programs recorded on one or more computer storage devices, each configured to perform the actions of the methods and systems described herein.

A further aspect of the disclosure is directed to system for performing a diagnostic procedure. The system includes a medical device configured for placement within a blood vessel and including a needle extendible from the medical device and into adventitia of the blood vessel, a light source disposed on a distal portion of the needle, and an ultrasound transducer disposed on a distal portion of the medical device. The system also includes a computing device including a processor and a memory storing instructions, which when executed by the processor, cause the computing device to: cause the light source to deliver a series of laser pulses to the adventitia of a blood vessel; detect ultrasonic waves emitted in response to the series of laser pulses; identify an elevated response within a portion of the detected ultrasonic waves compared to the remaining portion of the detected ultrasonic waves, where the identified elevated response is indicative of a presence of a nerve fascicle within the adventitia of the blood vessel; and determine a location of the nerve fascicle within the adventitia of the blood vessel. Other embodiments of this aspect include corresponding computer systems, apparatus, and computer programs recorded on one or more computer storage devices, each configured to perform the actions of the methods and systems described herein.

Yet a further aspect of the disclosure is directed to a method of assessing a target location for therapy. The method includes causing, by a computing device, a light source to deliver a first series of laser pulses to a first location along a wall of a blood vessel; causing, by the computing device, the light source to deliver a second series of laser pulses to a second location along the wall of the blood vessel; receiving, by the computing device, a first signal indicative of detected ultrasonic waves emitted in response to the first series of laser pulses; receiving, by the computing device, a second signal indicative of detected ultrasonic waves emitted in response to the second series of laser pulses; determining, by the computing device, a difference in time between emission of maximum amplitude of ultrasonic waves in response to the first series of laser pulses and emission of a maximum amplitude of ultrasonic waves in response to the second series of laser pulses; and calculating, by the computing device, a pulse wave velocity within the blood vessel based on a distance between the first location and the second location and the determined difference in time, where a pulse wave velocity in excess of a threshold is predictive of a response to therapy. Other embodiments of this aspect include corresponding computer systems, apparatus, and computer programs recorded on one or more computer storage devices, each configured to perform the actions of the methods and systems described herein.

Still a further aspect of the disclosure is directed to a method for performing a diagnostic procedure. The method includes causing, by a computing device, a light source to deliver a first series of laser pulses to an adventitia of a blood vessel; receiving, by the computing device a signal indicative of detected ultrasonic waves emitted in response to the first series of laser pulses; identifying, by the computing device, an elevated response within a portion of the detected ultrasonic waves compared to a remaining portion of the detected ultrasonic waves, where the identified elevated response is indicative of a presence of a nerve fascicle within the adventitia of the blood vessel; and determining, by the computing device, a location of the nerve fascicle based on the identified elevated response. Other embodiments of this aspect include corresponding computer systems, apparatus, and computer programs recorded on one or more computer storage devices, each configured to perform the actions of the methods and systems described herein.

Further disclosed herein is a system and method for performing a diagnostic procedure, the method including causing, a light source to deliver a first series and a second series of laser pulses to a first location and a second location along a wall of a blood vessel, wherein the method includes receiving a first signal indicative of detected ultrasonic waves emitted in response to the first series of laser pulses and receiving a second signal indicative of detected ultrasonic waves emitted in response to the second series of laser pulses, determining a difference in time between emission of maximum ultrasonic waves in response to the first series of laser pulses and emission of a maximum ultrasonic waves in response to the second series of laser pulses, calculating a pulse wave velocity within the blood vessel based on a distance between the first location and the second location and the determined difference in time.

### BRIEF DESCRIPTION OF THE DRAWINGS

Various aspects and embodiments of the disclosure are described hereinbelow with references to the drawings, wherein:

FIG. 1 is a schematic diagram of a system provided in accordance with the present disclosure;

FIG. 2 is a schematic view of a workstation of the system of FIG. 1;

FIG. 3 is a perspective view of a device of the system of FIG. 1 inserted within a patient;

FIG. 4A is a perspective view of a device undertaking diagnostic aspects of the disclosure;

FIG. 4B are graphs generated by the diagnostic aspects undertaken in FIG. 4A, in accordance with the disclosure;

FIG. 5A is a perspective view of a device undertaking another diagnostic aspect of the disclosure;

FIG. 5B is a graph generated by the diagnostic aspects undertaken in FIG. 5A, in accordance with the disclosure;

FIG. 5C is a perspective view of a device undertaking another diagnostic aspect of the disclosure;

FIG. 5D is a graph generated by the diagnostic aspects undertaken in FIG. 5D, in accordance with the disclosure;

FIG. 6 is a perspective view of a device undertaking another diagnostic aspect of the disclosure;

FIG. 7 is a flow chart describing a method in accordance with the disclosure; and

FIG. 8 is a flow chart describing a method in accordance with the disclosure.

### DETAILED DESCRIPTION

This disclosure is directed to diagnostic and, optionally, therapeutic systems and methods for pre-procedurally determining potential efficacy of, or peri-procedurally or post-procedurally assessing the efficacy of denervation or neuromodulation of nerves such as the sympathetic, or in certain embodiments, parasympathetic nerves, and in particular, unmyelinated nerve fibers in and around the blood vessels and other luminal tissue.

Chronic over-activation of the SNS has been associated with hypertension, particularly of the renal, hepatic, splanchnic, mesenteric, and other arteries of the body. To assess whether a patient is experiencing hypertension in one of these blood vessels a measurement of the pulse wave velocity (PWV) of the blood flowing through the blood vessel may be undertaken. An elevated PWV indicates that the muscles of the blood vessel are being signaled by the nerves surrounding the blood vessel resulting in a contraction of the blood vessel and an elevated PWV. While fluoroscopy and other imaging techniques have been developed to analyze PWV, improvements and more localized measurements are always desirable.

The diagnostic and therapeutic system of the disclosure utilizes a photoacoustic imaging system to calculate a PWV of fluid flowing within a blood vessel. In addition, the diagnostic and therapeutic system of the disclosure can employ photoacoustic imaging systems to identify a presence and/or location of sympathetic (or parasympathetic) nerves within the blood vessel.

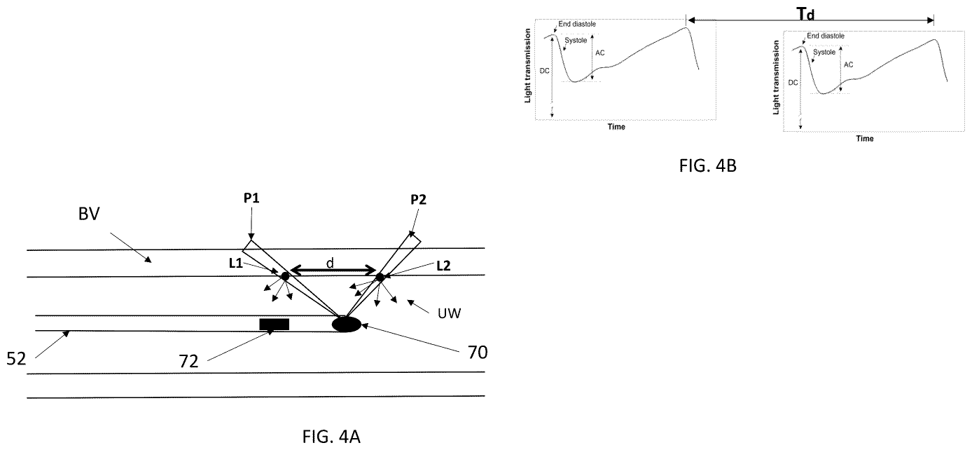

In accordance with aspects of the disclosure the diagnostic and, optionally, therapeutic system measures the PWV of fluid flowing within a blood vessel, such as a renal or hepatic artery, using photoacoustic signals which are similar to photoplethysmography (PPG) pulses. Pulses of light are emitted from one or more light sources having a wavelength between Ultra-Violet (UV) light and Near-Infrared (NIR) light (e.g., a wavelength of between approximately 200 nm and approximately 1400 nm). A first series of pulses of light is directed to a first location along the blood vessel wall and a second series of pulses of light is emitted towards a second location along the blood vessel wall. The second location is a distance from the first location. In some examples, the distance can be calculated based on a distance of the light emitter from the wall of the blood vessel and a known angle between the pulses of light. A portion of the light emitted by the light source is absorbed by certain biological molecules, including but not limited to hemoglobin, melanin, myoglobin, calcium, or other chromophores causing transient heating and expansion (e.g., transient thermoelastic expansion) of the affected biological molecules. The transient thermoelastic expansion of the affected biological molecules results in the emission of pressure wave signals or acoustic waves, such as ultrasonic waves (UW), which can be detected by an ultrasonic transducer. By focusing on the blood vessel wall with the emitted light, the ultrasonic waves that are emitted are actually from microvessels within the blood vessel but near the blood vessel wall on which the emitted light is directed. The ultrasonic waves detected by the ultrasonic transducer vary over time in accordance with the volume of arterial blood flow as the cardiac cycle proceeds through systole and diastole phases. The ultrasonic waves are converted to photoacoustic signals by the transducer and correlate to volumetric changes associated with pulsatile arterial blood flow. A graph or plot can be generated from the photoacoustic signals illustrating an increase and decrease of light absorption due to changes in blood volume over time. (See FIG. 4B) These graphs can be generated for the first and the second locations and a time difference between observes of a maximum or a minimum at the first location as compared to the second location (a calculated distance from each other) enable the calculation of the PWV of the blood vessel.

In addition to or instead of calculating PWV within the blood vessel to identify candidates for denervation or the efficacy of denervation, the therapeutic system may utilize photoacoustic signals to identify, image, and/or map nerve fascicles associated with nerves within adventitia of the blood vessel. Certain molecules of the nerve fascicles, such as calcium channels, are sensitive to photoacoustic imaging. The calcium channels of the nerve fascicles generate an elevated response or a spike in the ultrasonic waves from absorption of the light emitted from the light source as compared to the ultrasonic waves generated by surround tissue. In this manner, the location of nerve fascicles in the adventitia of the blood vessel can be identified relative to the ultrasonic transducer and, a map of one or more nerve fascicles within the target tissue can be generated. As can be appreciated, with the location of the nerve fascicles within the blood vessel identified, denervation can be targeted to specific portions of the blood vessel to minimize damage to surrounding tissue and increase the likelihood of successful denervation.

In some implementations, after the application of denervation therapy, the PWV in the blood vessel may be remeasured to determine the efficacy of the treatment. In this manner, a PWV that is lower than the PWV measured before treatment, or that is below a predetermined threshold, indicates that the application of therapy has been successful. Conversely, a PWV remains approximately the same as the PWV measured before treatment, or that is above the predetermined threshold, indicates that the application of therapy has not been successful, and a re-application of therapy may be required.

Similarly, if desired, an inquiry relating to whether a previously mapped nerve fascicle has been denervated can be undertaken following a denervation procedure. Where nerve fascicles have been mapped, a post denervation photo acoustic interrogation can be undertaken and an elevated response in the UW or the photoacoustic signal correlated thereto indicates that the application of therapy has not been successful. In contrast, the absence of an elevated response in the UW or photoacoustic signal, for example as compared to the UW or photoacoustic signal from surrounding tissue, indicates successful denervation. If re-application of therapy is required, the subsequent interrogation can be used to update the map of the locations of the nerves and the position at which the denervation therapy is applied and or other parameters of the denervation (e.g., more energy, less energy, more chemical agent, less chemical agent, more cryogen, less cryogen, etc.) can also be adjusted.

In some implementations, the diagnostic and, optionally, therapeutic system may include one or more telescoping needles configured to penetrate the target tissue and deliver light directly within the adventitia of the blood vessel. It is envisioned that the telescoping needle may be or include a microlaser (e.g., a needle including a light source on the distal tip) or a needle including a fiber optic cable or other light transmitting element disposed therein. The telescoping needles may be individually deployed and may be manually (or automatically) controllable by the system or user or may be constructed of a shape memory material. It is envisioned that the one or more telescoping needles may be radiopaque, such that the one or more telescoping needles may be identified using fluoroscopy, during an angiogram, or using any suitable imaging modality.

In some examples, the diagnostic and therapeutic system may include one or more therapeutic devices for delivering denervation therapy within adventitia of the blood vessel that is capable of applying one or more of a variety of therapeutic modalities without departing from the scope of the present disclosure. Therapeutic modalities considered within the scope of this disclosure include monopolar or bipolar radiofrequency, microwave, cryogenic, ultrasound, chemical, electrical, direct heat, radiation, and other yet to be developed modalities. Any of these therapy modalities may be incorporated into a therapeutic device, such as a catheter, which is configured for navigation to a desired location within the patient. In embodiments, the energy delivery elements and/or therapy delivery portion of the therapy catheter may be disposed adjacent to the ultrasound transducer.

A catheter configured to deliver one or more of these diagnostic or therapeutic modalities may be percutaneously navigated, for example via the femoral artery, radial artery, brachial artery, or the like to reach the blood vessels of the aorta including the renal arteries, celiac artery, hepatic arteries, splanchnic arteries, mesenteric arteries, and other arteries that are enervated with sympathetic nerves or are proximate one or more sympathetic nerve ganglia. As will be appreciated, for certain procedures more than one of these arteries may be denervated without departing from the scope of the disclosure. For instance, two arteries, such as the renal artery and the hepatic artery, may be denervated. The catheter may also be laparoscopically placed in one or more of the above-identified blood vessels, or another luminal tissue without departing from the scope of the present disclosure. In embodiments, the system may generate a notification or other indicator (e.g., audible tone, haptics, visual indicators such as lights, colors, text, etc.), that the catheter is adjacent nerve fascicles identified within the target tissue. In some implementations, the system may automatically initiate, or inhibit, the delivery of denervation therapy to the target tissue based upon the determination that the catheter is adjacent nerve fascicles.

For ease of description, much of the following description focuses on implementations of electrical stimulation and RF denervation. Those having skill in the art will recognize that the methods and systems described herein may employ any of the therapy and/or neurostimulation modalities described herein. Similarly, the following description focuses on navigation to and application of neurostimulation and/or therapy to the renal artery to denervate sympathetic or, in certain embodiments, parasympathetic, nerves in, around, and proximate the renal arteries. However, the present disclosure is not so limited and can be employed for denervating nerves accessible via any blood vessel described herein or other luminal tissue (e.g., a bile duct).

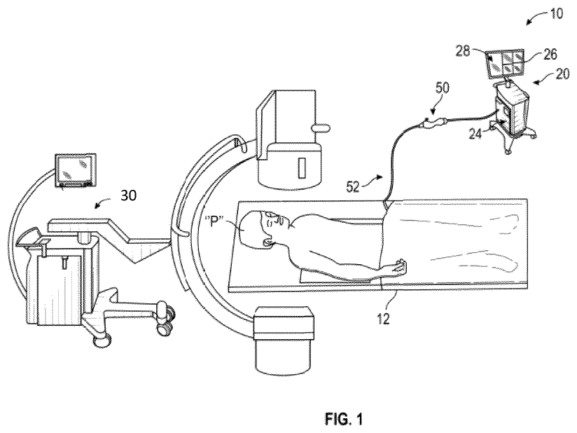

Turning now to the drawings, FIG. 1 illustrates a system in accordance with the present disclosure and generally identified by reference numeral 10. As will be described in further detail hereinbelow, the system 10 enables navigation of a medical device 50 to a desired location within the patient's anatomy (e.g., the patient's renal artery), delivery of pulses of light to tissue within the renal artery, detection of ultrasonic waves (UW) generated as a result of the delivery of pulses of light to the tissue within the renal artery, conversion of the UW to photoacoustic signals, calculating a Pulse Wave Velocity (PWV) of fluid flowing within the blood vessel tissue, identification of a location of one or more nerve fascicles within adventitia of the renal artery using photoacoustic signals, adjustments of a position of the therapeutic device within the renal artery based upon the photoacoustic signals, application of denervation therapy to the tissue within the renal artery to denervate sympathetic nerves within the tissue, delivery of pulses of light to the tissue within the renal artery and detection of a photoacoustic signal to assess the efficacy of the denervation therapy, and combinations thereof.

The system 10 includes a workstation 20, a medical device 50 operably coupled to the workstation 20, and an imaging device 14, which may be operably coupled to the workstation 20. The patient “P” is shown lying on an operating table 12 with the medical device 50 inserted through a portion of the patient's femoral artery, although it is contemplated that the medical device 50 may be inserted into any suitable portion of the patient's vascular network that is in fluid communication with a desired blood vessel for therapy, or another luminal network. Although generally described as having one medical device 50, it is envisioned that the system 10 may employ any suitable number of medical devices 50. The medical devices 50 may employ the same or different therapy modalities may and be operably coupled to the workstation 20. Further, the medical device 50 may employ a guidewire 64 or a guide catheter 62 (FIG. 4) without departing from the scope of the disclosure.

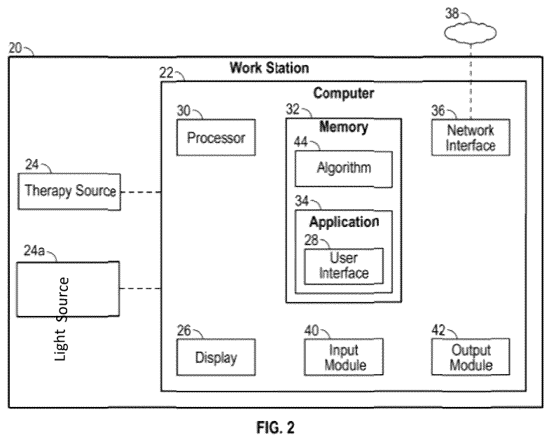

Continuing with FIG. 1 and with additional reference to FIG. 2, the workstation 20 includes a computer 22 and a therapy source 24 (e.g., an RF generator, a microwave generator, an ultrasound generator, a cryogenic medium source, a chemical source, etc.) operably coupled to the computer 22. The computer is coupled to a display 26 that is configured to display one or more user interfaces 28. The computer 22 may be a desktop computer or a tower configuration with display 26 or may include a laptop computer or other computing device. The computer 22 includes a processor 30 which executes software stored in a memory 32. The memory 32 may store one or more applications 34 and/or algorithms 44 to be executed by the processor 30. A network interface 36 enables the workstation 20 to communicate with a variety of other devices and systems via the internet. The network interface 36 may connect the workstation 20 to the Internet via a wired or wireless connection. Additionally, or alternatively, the communication may be via an ad hoc Bluetooth® or wireless network enabling communication with a wide-area network (WAN) and/or a local area network (LAN). The network interface 36 may connect to the Internet via one or more gateways, routers, and network address translation (NAT) devices. The network interface 36 may communicate with a cloud storage system 38, in which further data, image data, and/or videos may be stored. The cloud storage system 38 may be remote from or on the premises of the hospital such as in a control or hospital information technology room. It is envisioned that the cloud storage system 38 could also serve as a host for more robust analysis of acquired images (e.g., fluoroscopic, computed tomography (CT), magnetic resonance imaging (MRI), cone-beam computed tomography (CBCT), etc.), data, etc. (e.g., additional or reinforcement data for analysis and/or comparison). An input module 40 receives inputs from an input device such as a keyboard, a mouse, voice commands, an energy source controller (e.g., a foot pedal or handheld remote-control device that enables the clinician to initiate, terminate, and optionally, adjust various operational characteristics of the therapy source 24, including, but not limited to, power delivery), amongst others. An output module 42 connects the processor 30 and the memory 32 to a variety of output devices such as the display 26. In embodiments, the display 26 may be a touchscreen display.

The therapy source 24 generates and/or outputs one or more of RF energy (monopolar or bipolar), microwave energy, ultrasound energy, cryogenic medium, or chemical ablation medium via an automated control algorithm 44 stored on the memory 32 and/or under the control of a clinician. As can be appreciated, the therapy generated or output by the therapy source 24 changes a temperature of the tissue (e.g., increases or decreased the temperature) to achieve the desired denervation of the nerves. The therapy source 24 may be configured to produce a selected modality and magnitude of energy and/or therapy for delivery to the treatment site via the medical device 50, as will be described in further detail hereinbelow. The therapy source 24 may monitor voltage and current applied to target tissue via the medical device 50 and may monitor the temperature of the target tissue or tissue proximate the target tissue, and/or a portion of the medical device 50.

The workstation 20 may also include a light source 24a, which is capable of generating one or more sources of light for transmission through a fiber-optic cable or other means to the medical device 50 for use in the methods described herein for measurement of PWV, identification of location of nerve fascicles, and the assessment of efficacy of therapy.

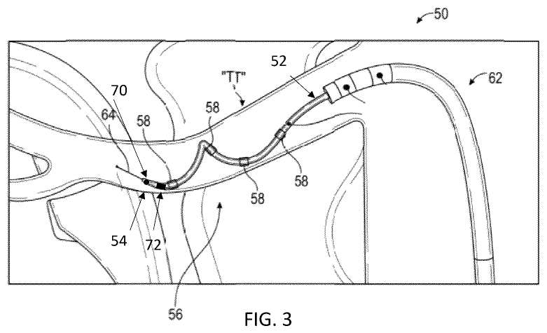

FIG. 3 depicts an embodiment of a medical device 50 in accordance with the disclosure. The medical device 50 includes an elongated shaft 52 having a handle (not shown) disposed on a proximal end of the elongate shaft 52. The elongate shaft 52 of the medical device 50 is configured to be advanced within a portion of the patient's vasculature, such as a femoral artery or other suitable portion of patient's vascular network that is in fluid communication with the patient's renal artery. As depicted in FIG. 3, the elongate shaft 52 may be configured to be received within a portion of a guide catheter or guide sheath (such as a 6F guide catheter) 62 that is utilized to navigate the medical device 50 to a desired location at which point the guide catheter 62 is retracted to uncover the therapeutic portion 56 of the medical device 50 which in the embodiment shown includes a plurality of monopolar electrodes 58. The elongated shaft 52 of the medical device 50 may further include an aperture (not shown) that is configured to slidably receive a guidewire 64 over which the medical device 50, either alone or in combination with the guide catheter 62, are advanced. In this manner, the guidewire 64 is utilized to guide the medical device 50 to the target tissue using over-the-wire (OTW) or rapid exchange (RX) techniques, at which point the guide wire may be partially or fully removed from the medical device 50. A distal end portion 54 of the medical device 50 includes one or more light emitters 70 and an ultrasound transducer 72.

Continuing with FIGS. 3 and 4, in an embodiment, a light emitter 70 is disposed on the distal end portion 54 of the medical device 50 that is configured to emit light towards a wall of the target tissue. The light emitter 70 may be any suitable light source capable of emitting light having a wavelength between Ultra-Violet (UV) light and Near-Infrared (NIR) light (e.g., a wavelength of between approximately 200 and 1400 nm) without departing from the scope of the present disclosure. In embodiments, the light emitter 70 may be a laser (e.g., a Light Emitting Diode (LED), a laser diode, etc.) configured to emit light having a wavelength between approximately 600 nm and 700 nm, although any suitable light source and/or wavelength capable of penetrating tissue and being absorbed by hemoglobin may be utilized without departing from the scope of the present disclosure. This light emitter 70 may emit light at a power of approximately 10 mJ to approximately 100 mJ. As described above, light emitted from the light emitter 70 (e.g., a laser) is focused on the wall of the blood vessel. By focusing on the blood vessel wall, UW emissions from the biological molecules (e.g., hemoglobin) within microvessels located at or near the blood vessel wall can be detected. It is envisioned that the light emitter 70 may be operably coupled to the light source 24a via a light transmitting conduit (e.g., fiber optic cable, etc.) operably coupled to the medical device 50. In embodiments, the light emitter 70 may be a light emitting diode (LED), a bi-directional laser mounted on a distal portion of the medical device 50 and in electrical communication with the workstation 20 to provide energy for the illumination of the blood vessel as described herein below.

A portion of the light emitted from the light emitter 70 is absorbed by biological molecules, such as hemoglobin, flowing within the microvessels at nor near the wall of the blood vessel causing transient heating and expansion (e.g., transient thermoelastic expansion) of the affected biological tissue. The transient thermoelastic expansion of the affected biological molecules results in the emission of acoustic waves, such as ultrasonic waves (UW), which are detected by the ultrasound transducer 72. The ultrasound transducer 72 is disposed adjacent to the light emitter 70 and at a known distance therefrom. The medical device 50 may include a plurality of ultrasonic transducers 72 disposed on the elongated shaft 52. The ultrasound transducer 72 is operably coupled to the workstation 20, which correlates the detected ultrasonic waves to a measurement of light-absorption due to a systolic increase in arterial blood volume (e.g., a photoacoustic signal). A plot or graph can be generated from the photoacoustic signals illustrating an increase and decrease of light absorption due to changes in blood volume over the length of a pulse of light emitted from the light emitter 70, from which maximal and minimal values of the photoacoustic signals can be identified.

To calculate the PWV of fluid flowing within the blood vessel, photoacoustic signals are measured by directing light from the light emitter 70 at two different locations along the length of the blood vessel. The light emitter 70 emits a first series of pulses of light “P1” at a first angle towards a first location “L1” along the wall of the blood vessel. The light emitter 70 also emits a second series of pulses of light “P2” at a second angle towards a second location “L2” along the wall of the blood vessel. The individual pulses of light “P1” may have a duration of a picosecond and the individual pulses of light “P2” may have a duration of a nanosecond, although it is contemplated that the first and second pulses of light “P1,” “P2” may be of any suitable duration without departing from the scope of the present disclosure so long as the pulses “P1” and “P2” do not have the same duration. A portion of the light emitted from the first and second series of pulses of light “P1,” “P2” is absorbed by hemoglobin or other biologic molecules in the microvessels in or near the wall of the blood vessel causing the emission of resulting ultrasonic waves (UW). The UW are detected by the ultrasound transducer 72. The transducer converts the UW to photoacoustic signals that the software application stored on the memory 32 of the workstation 20 analyzes and, calculates a PWV using the geometry described above and depicted in FIGS. 4A and 4B. The software application identifies a maximum or a minimum amplitude value of the photoacoustic signals measured by the ultrasound transducer 72 at each of the first and second locations “L1,” “L2.” A time difference between the identified maximum or minimum amplitude values as observed at L1 compared to L2 is calculated. As an example, and with reference to FIG. 4B, the photoacoustic signal is graphed and the difference between the peak observed at L1 and L2 is a time (Td).

A distance of the light emitter 70 from the blood vessel (BV) wall may be determined by analysis of images captured by a camera (not shown) associated with the medical device 50, intraprocedural CT imaging, fluoroscopy, or other imaging modalities. To assist in this the light emitter 70 or the ultrasound transducer 72 may be radiopaque, or a radiopaque marker may be incorporated into the distal portion of the therapy device 50. A distance between the first and second locations “L1,” “L2” is calculated using a known angle at which the first and second pulses of light “P1,” “P2” are emitted from the light emitter 70 and the identified distance between the light emitter 70 and the walls of the blood vessel. Specifically, with the distance from the light emitter 70 from the blood vessel wall and the angle between light pulses P1 and P2 known, a distance from the light emitter 70 to locations L1 and L2 are solved for using basic trigonometry, and a resulting distance d can thus be solved for. The software application calculates the PWV of the fluid flowing within the target tissue using the calculated distance between the first and second locations “L1,” “L2” and the calculated time difference (Td) between the identified maximum or minimum values of the photoacoustic signals (FIG. 4B). In this manner, in situ PWV calculations can be made of specific blood vessels within the body without requiring any specific level of blood perfusion within that blood vessel. Use of the distance d of the medical device 50 from the blood vessel wall for calculation of the distance between L1 and L2 is enabled, in part, because the light emitter 70 focuses on the blood vessel wall and the transducer 72 detects the UW emissions from the microvessels at or near the surface of the blood vessel.

In a further aspect of the disclosure, the location and/or orientation of the light emitter 70 and the ultrasound transducer 72 relative to the blood vessel can be adjusted. In this manner, a number of calculations of PWV may be undertaken at different points along the blood vessel to determine if there are differences in PWV and therewith differences in the contracted state of the blood vessel. Locations where PWV is higher maybe more likely to benefit from denervation than locations which have a lower PWV. Thus, a PWV map along a length of the blood vessel can be assembled and compared to one or more thresholds, where a PWV less than the one or more thresholds indicates that the tissue would not be good candidates for denervation whereas a calculated PWV greater than the one or more thresholds indicates that the tissue would be good candidates for denervation. As will be appreciated, the PWV determination can be undertaken multiple times both to determine whether the patient is a good candidate for denervation, and to identify portions of the blood vessel most likely to benefit from denervation.

It is envisioned that the calculated PWV for each location can be monitored by a control algorithm 44 stored on the workstation 20, with the location and results stored in the memory 32. The stored PWV can be compared to predetermined thresholds stored in the memory 32 or to other calculated PWV's stored in the memory to aid the clinician in identifying the optimal locations and/or orientation of the therapeutic portion 56 of the medical device 50 relative to the target tissue. A look-up table of data of predetermined thresholds may be saved within the memory 32 and accessed by the control algorithm 44 or other suitable application stored on the memory 32 during the procedure and alerts may be presented on the user interface 28.

In addition to identifying candidate tissue for denervation, it is envisioned that the method of calculating PWV of fluid flowing within target tissue described herein may be utilized to analyze the efficacy of denervation therapy. In this manner, after the application of therapy from the therapy source 24 to the target tissue, the first and second pulses of light “P1,” “P2” are once again emitted from the light emitter 70 and the PWV is calculated. As can be appreciated, successful denervation would result in a lower PWV as compared to the PWV calculated before the application of therapy. Similar to the method described hereinabove with respect to identifying potential candidates for denervation, the PWV can be calculated and compared to one or more thresholds. A post therapy PWV that is less than the one or more thresholds is indicative of successful denervation whereas a PWV that is greater than the one or more thresholds suggests further denervation may be required.

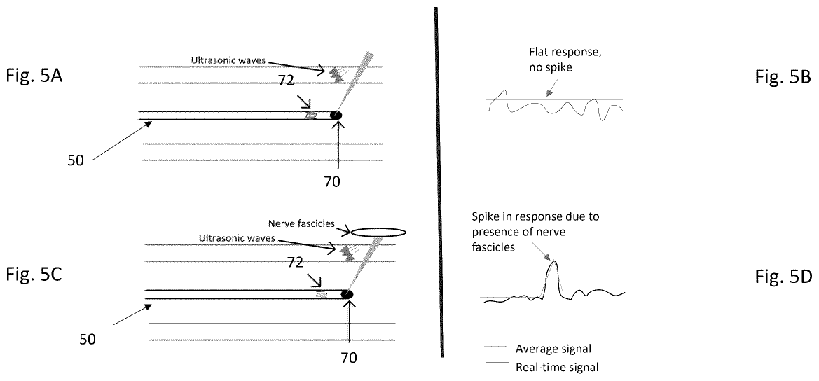

With reference to FIGS. 5A-5D, in addition to, or in lieu of, calculating PWV, it is envisioned that the system 10 may utilize photoacoustic signals to identify, or map nerve fascicles within adventitia of the target tissue. As can be appreciated, identification, imaging, or mapping the nerve fascicles may be performed during the process of calculating PWV or after confirming that a patient is a candidate for denervation using PWV calculations, or after the application of therapy to determine the efficacy of denervation of the target tissue.

Certain molecules of the nerve fascicles, such as calcium channels, are sensitive to photoacoustic imaging. The calcium channels of the nerve fascicles generate an elevated response or a spike in the ultrasonic waves generated by the absorption of light emitted from the light emitter 70 as compared to the ultrasonic waves generated by absorption of light by surrounding tissue and biological molecules, such as hemoglobin. FIGS. 5A and 5C depict a similar arrangement of FIG. 4A with the medical device 50 within a blood vessel (BV) and emitting pulses of light (e.g., P1 or P2 described above). As with the PWV calculation above, ultrasonic waves (UW) are generated as a response to the transmission and absorption of the pulses of light. FIGS. 5B and 5D depict the response to the pulses of light as captured by the ultrasonic transducer 72. As can be seen graphically, in the presence of nerve fascicles the observed response in generated (UW) (FIG. 5D) is much greater than in their absence (FIG. 5C). In this manner, the location of nerve fascicles in the adventitia of the blood vessel can be identified relative to medical device 50 and using the ultrasonic transducer a map of the locations of the nerve fascicles within the blood vessel can be generated. As can be appreciated, with the location of the nerve fascicles within the blood vessel identified, denervation can be targeted to specific portion of the blood vessel to minimize damage to surrounding tissue (e.g., minimize the margin) and increase the likelihood of successful denervation. The light emitted from the light emitter 70 when identifying nerve fascicles may include any wavelength between UV light and NIR light (e.g., a wavelength of between approximately 200 and 1400 nm). In embodiment, the frequency of the light emitted from the light emitter 70 may be swept from low frequency to high frequency and the power of the emitted light may

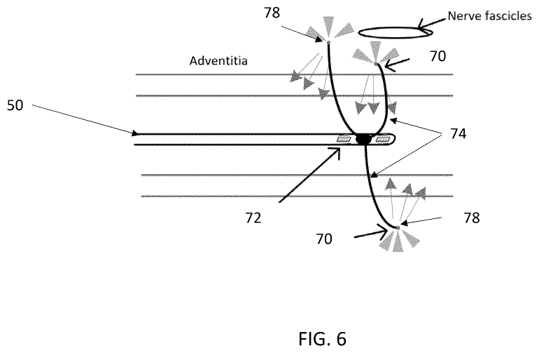

In accordance with a further aspect of the disclosure, and with reference to FIG. 6, one or more telescoping probes 74 may be disposed within the distal end portion 54 of the medical device 50. The telescoping probe 74 may be disposed within a hollow interior portion (not shown) of the medical device 50 and is deployable through an aperture formed in the medical device 50. A distal tip 78 of the telescoping probe 74 includes a light emitter 70 as described above, which in embodiments, may be a microlaser. It is contemplated that the telescoping probe 74 may be operably coupled to the light source 24a via a light transmitting conduit (e.g., fiber optic cable, etc.) that terminates at the distal tip 78 of the telescoping probe 74 (e.g., within the hollow interior portion of the therapeutic device, outside the patient's body, etc.) without departing from the scope of the present disclosure.

The telescoping probe 74 is deployable from a retracted position to one or more extended positions that penetrates the wall of the blood vessel such that the distal tip 78 is disposed within the adventitia of the blood vessel. Placed in the adventitia the distal tip 78, and therefore, the light emitter 70, may more accurately direct light towards the nerve fascicles within the adventitia as compared to the light emitter 70 being disposed within the lumen of the target tissue. In this manner, delivering light directly within the adventitia generates higher resolution photoacoustic signals and better identification and/or mapping of the nerve fascicles within the adventitia as compared to the light emitter 70 being disposed within the lumen of the target tissue. As can be appreciated, by advancing the distal tip 78 of the telescoping probe 74 within the adventitia, the power of the light emitted from the light emitter 70 may be reduced as compared to the power of the light emitted from the light emitter 70 disposed within the lumen of the renal artery. The telescoping probes 74 may be manually or automatically manipulated in extension, retraction, curvature, etc. using any suitable means, and in embodiments, may be formed from a shape memory material where exit from the medical device 50 frees the shape memory alloy to achieve the desired shape for penetrating the tissue wall and the adventitia. In one non-limiting embodiment, the telescoping probes 74 include an outer dimension of approximately 25-30 gauge (e.g., approximately 0.53 mm to 0.42 mm).

Although generally described as having a single telescoping probe 74, it is envisioned that the medical device 50 may include any number of telescoping probes 74 which may be directed in various directions to identify nerve fascicles more quickly within the adventitia of the target tissue. It is envisioned that each of the telescoping probes 74 may be manipulatable or navigated independently of one another or manipulated in unison.

In embodiments, when disposed in the retracted position (e.g., not penetrating the tissue wall), the telescoping probe 74 may be utilized to deliver the first and second pulses of light “P1,” “P2” to the target tissue to calculate the PWV of the target tissue. Once the patient is confirmed as a candidate for denervation by calculating PWV, the telescoping probe 74 may remain in the retracted position and emit pulses of light from the distal tip 78 to identify the location of nerve fascicles within the adventitia, as described in further detail hereinabove. Once nerve fascicles have been identified as present within the adventitia, the telescopic probe 74 may be transitioned to the extended position within the adventitia of the candidate tissue to identify the position of nerve fascicles more accurately.

After the application of denervation therapy, the target tissue may be re-illuminated to determine the efficacy of the therapy. As noted above this may be in the form of a second PWV calculation where are sufficiently reduced PWV indicates a successful denervation or a second interrogation of the adventitia searching for the presence of the nerve fascicles. With regards to the interrogation of the adventitia, an elevated response in the photoacoustic signals compared to the photoacoustic signals from surrounding tissue indicates that the application of therapy has not been successful. In contrast, the absence of an elevated response or spike in the photoacoustic signals compared to the photoacoustic signals from surrounding tissue indicates successful denervation. Alternatively, the response prior to denervation can be compared to the post denervation response and a change more than a threshold may be used to indicate the success of the denervation.

As above, if re-application of therapy is required, the interrogation signals may be used to update the position at which the denervation therapy is applied and/or adjust the delivery of therapy (e.g., more energy, less energy, more chemical agent, less chemical agent, more cryogen, less cryogen, etc.). In embodiments where one or more telescoping probes 66 are deployed, the efficacy of the denervation therapy can be determined by emitting pulses of light in the same location and monitoring the photoacoustic signal, which can accurately identify whether specific nerve fascicles have been destroyed. Similar to that described herein above, it is envisioned that the photoacoustic signals generated when imaging or mapping nerve fascicles may be monitored by the control algorithm 44 stored on the computing device, with the location and results of the application of light and/or photoacoustic signal responses stored in the memory 32. The stored photoacoustic responses signals can be compared to predetermined thresholds stored in the memory or to other photoacoustic signals stored in the memory to aid the clinician in identifying the optimal locations and/or orientation of the therapeutic portion 56 of the medical device 50 relative to the target tissue. A look-up table of data or predetermined thresholds may be saved within the memory 32 and accessed by the control algorithm 44 or other suitable application stored on the memory 32 during the procedure and alerts may be presented on the user interface 28.

In embodiments, a map of the location of nerve fascicles within the adventitia may be generated and displayed on the user interface 28. Labels of each identified nerve fascicle may be automatically or manually applied based upon the monitored photoacoustic signals and displayed for example on the periprocedural images used to determine the distance of the medical device 50 from the blood vessel. In this manner, as denervation therapy is applied to each nerve fascicle or area surrounding the identified nerve fascicles, successful denervation for each nerve fascicle can be indicated on the user interface 28. Identified nerve fascicles that have not yet been denervated or require additional therapy can also be indicated on the user interface 28, with additional information associated with each identified nerve fascicle being displayed by toggling or otherwise engaging the desired nerve fascicle displayed on the user interface 28. In one non-limiting embodiment, the location of nerve fascicles may be overlaid on pre-procedural or peri-procedural images, such as Computed Tomography (CT) images, a 3D model generated from pre-procedural or peri-procedural images, fluoroscopic images, Cone Beam CT, Ultrasound, MRI, etc. amongst other imaging modalities.

It is envisioned that upon identification of nerve fascicles within candidate target tissue, the control algorithm 44 may generate a notification or other indicator (e.g., an audible tone, haptics, visual indicators such as lights, colors, text, etc.), that the therapeutic portion 56 of the medical device 50 is adjacent nerve fascicles identified within the target tissue. In embodiments, the control algorithm 44 may automatically initiate, or terminate, the delivery of denervation therapy to the target tissue based upon the determination that the therapeutic portion 56 of the medical device 50 is adjacent nerve fascicles.

In embodiments where the medical device 50 is an RF ablation catheter, the therapeutic portion 56 includes one or more electrodes 58 disposed on the shaft 52 that are configured to apply denervation therapy to the target tissue. It is envisioned that the one or more electrodes 58 may be disposed in spaced relation to each other and configured to contact the blood vessel walls suitable configuration, such as a helical, expanded configuration or by manipulating the therapeutic portion 56 to contact tissue walls (e.g., in the case of a linear arrangement). In one non-limiting embodiment, the therapeutic portion 56 is configured to be transitioned from an initial, undeployed state having a generally linear profile, to a second, deployed or expanded configuration, where the therapeutic portion 56 forms a generally spiral and/or helical configuration (FIG. 3) for delivering therapy at the treatment site and providing therapeutically-effective electrically and/or thermally induced renal neuromodulation. In this manner, when in the second, expanded configuration, the therapeutic assembly 56 is pressed against or otherwise contracts the walls of the patient's vasculature. Although generally described as transitioning to a spiral and/or helical configuration, it is envisioned that the therapeutic assembly 56 may be deployed to any suitable shape. In embodiments, the therapeutic portion 56 of the medical device may be capable of being placed in any suitable numbers of configurations depending upon the design needs of the medical device 50 or the type of procedure being performed. As shown herein, the medical device 50 includes four electrodes 58. However, the present disclosure is not so limited and the medical device 50 may have more or fewer electrodes 58 without departing from the scope of the present disclosure. One of skill in the art will recognize that the electrodes 58 may be replaced with ultrasound transducers, microwave antennae, ports for delivery of cryoablation medium or chemical medium and other implements and/or ablation and denervation modalities without departing from the scope of the present disclosure

As illustrated in the figures, the electrodes 58 are disposed in spaced relation to one another along a length of the medical device 50 forming the therapeutic portion 60. As will be appreciated, these electrodes 58 are in communication with the therapy source 24 which produces, for example, monopolar RF energy to denervate the sympathetic nerves of the relevant blood vessel. Additionally, or alternatively, the electrodes 58 may delivery RF energy independently of one another (e.g., monopolar), simultaneously, selectively, sequentially, and/or between any desired combination of the electrodes 58 (e.g., bipolar).

In one embodiment, the medical device 50 may be a cryotherapy device where the therapeutic portion 56 may include one therapy delivery element, such as an occlusive balloon, a non-occlusive balloon, or other balloon permitting the flow of blood, etc. In this embodiment, the therapy source 24 may include a cryogen or coolant source or means to generate a cryogen. It is envisioned that the medical device 50 may be a microwave energy device where the therapeutic portion 56 may include one or more therapy delivery elements, such as a microwave antenna. In this embodiment, the therapy source 24 may be a microwave energy generator that is operably coupled to the microwave antenna. It is contemplated that the therapeutic device may be an ultrasound device where the therapeutic portion 56 may include one or more therapy delivery elements, such as an ultrasound transducer, etc. (which may be the same or in addition to ultrasound transducer 72). In this embodiment, the therapy source 24 may be a radio-frequency energy generator or the like that is operably coupled to the ultrasound transducer. In embodiments, the medical device 50 may be a chemical denervation device where the therapeutic portion 56 may include one or more cannulas or needles for the administration of a chemical denervation agent. In this embodiment, the therapy source 24 may be a chemical denervation agent source that is operably coupled to the therapeutic portion 60. Those having skill in the art will recognize that the medical device 50, the therapeutic portion 60, and the therapy source 24 may be any suitable combination of devices capable of performing a denervation procedure.

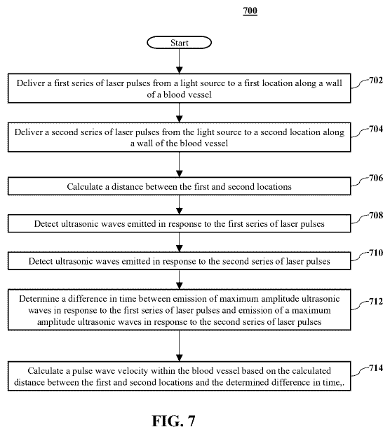

Though described in detail above, FIG. 7 sets forth a basic method of employing the systems described herein to determine the PWV of blood flow through a blood vessel. As will be appreciated the method of claim 7 follows the navigation of a medical device 50 to a desired location with the patient (e.g., within a renal, hepatic, splanchnic, or mesenteric artery). Once positioned within the desired blood vessel, the method 700 begins at step 702 with light emitter 70 delivering a first series of pules of laser light onto a first location along the wall of the blood vessel. A second series of pulses of laser light are delivered by the light emitter 70 to a second location along the wall of the blood vessel at step 704. As will be appreciated steps 702 and 704 may occur simultaneously or at overlapping times without departing from the scope of the disclosure. At step 706 the application 34 on computer 22 calculates a distance between the first location and the second location. As noted above, those may be done employing basic trigonometry, a determination of a distance of the therapeutic device from the wall of the blood vessel, particularly at the light emitter 70, and knowing the angles at which the first and second series of light pulses are directed towards the first and second locations relative to the therapeutic device. The first and second series of laser light pulses are absorbed by biologic tissues such as hemoglobin and cause the emission of ultrasonic waves. The ultrasonic waves emitted in response to the first series of laser light pulses is detected by the ultrasound transducer 72 at step 708. Similarly, the ultrasonic waves emitted in response to the second series of laser light pulses is detected by the ultrasound transducer 72 at step 710. The magnitude of the emissions of the ultrasonic waves is analyzed, for example via an application 34 stored in the memory 32 of the workstation 20 to determine a time at which a maximum emission of ultrasonic waves occurs at the first location and the second location, and a difference in time is calculated at step 712. At step 714, based on the time difference calculated by the application stored in the memory 32 of the workstation 20 at step 712 and the distance calculated at step 706, a pulse wave velocity is calculated for the blood vessel in which the medical device 50 is located. Though described here as analyzing the ultrasonic waves emitted in response to the interrogation by the light emitter 70, as noted herein above, the transducer 72 converts the detected UW waves into photoacoustic signals (e.g., FIG. 4B), which may be analyzed as described elsewhere herein without departing from the scope of method 700. Further, though steps 702, 704, 708, and 710 are described as being performed by the light emitter 70 and the transducer 72, steps of method 700 may be performed by portions of the workstation 22, such as the light source 24a and the application 34 stored in memory 32.

As noted above, a PWV in excess of a threshold is indicative of a patient and a blood vessel that can benefit from denervation therapy to reduce hypertension and other conditions of the body. Further, as described herein above, the method 700 may be performed multiple times, before denervation and after denervation to assess whether the denervation of the blood vessel has been successful. Successful denervation should result in a change of pulse wave velocity (a reduction) in excess of a threshold. Further, the PWV determination of method 700 may be employed during a denervation procedure, and upon sensing a change in PWV or a rate of change in PWV in excess of a given threshold the denervation procedure (e.g., application of RF energy) may be stopped as the change in PWV is an indication that the denervation has been successful. Still further, a lack of change in PWV is indicative of either no nerves being present at the location and suggesting that the therapeutic device should be moved, or that the nerves at that location lie deeper in the adventitia of the blood vessel and the denervation parameters (e.g., power, duration, frequency, etc.) can be altered and the denervation repeated until either a maximum has been reached or a successful denervation has been achieved.

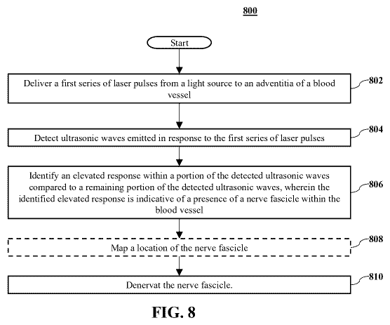

Another aspect of the disclosure described above is utilizing the medical device 50 to determine the locations of nerves and particularly nerve fascicles within the adventitia of the blood vessel. Method 800, described in connection with FIG. 8 sets forth such a diagnostic method. At step 802, a first series of laser pulses are emitted from the light emitter 70 and directed at the adventitia of the blood vessel. Ultrasonic waves are emitted in response to the absorption of the first series of laser pulses, the emitted ultrasonic waves are detected at step 804 by the ultrasound transducer 72 at step 804 and transmitted to the workstation 20 and computer 22. An application 34 stored in the memory 32 of the workstation 20 analyzes the detected ultrasonic waves to identify an elevated response within a portion of the detected ultrasonic waves at step 806. The application 34 may optionally map the locations from which the elevated response is detected as locations of nerve fascicles at step 808. With the nerve fascicles detected and optionally mapped, the medical device 50 can then be employed to denervate the blood vessel, and particularly the detected nerve fascicle at step 810. Though described herein above as analyzing the ultrasonic waves emitted in response to the interrogation of by the light emitter 70, as noted herein above, the transducer 72 converts the detected UW into photoacoustic signals (e.g., similar to PPG signals), which may be analyzed as described elsewhere herein without departing from the scope of method 800. Further, steps 802-808, can be performed by portions of the workstation, such as light source 24a and the application 34 stored in memory 32 and step 810 can be performed by the therapy source 24.

As described herein, method 800 may be used alone or in combination with method 700. In one embodiment, method 700 is employed to ensure that the patient is a candidate for denervation therapy and that the blood vessel in question is experiencing hypertension likely caused by over-activation of the SNS. Once confirmed, method 800 can be employed to determine where in the blood vessel the nerve fascicles are located in the blood vessel and denervation of the detected nerve fascicles. Method 700 may then be employed to confirm that the denervation has been successful. Additionally or alternatively, confirmation of denervation can be undertaken by employing steps 802-806, where if the denervation is successful then no elevated response, or an elevated response has been reduced by some threshold indicates a successful denervation.

Though specific methods 800 and 700 are described herein, those of skill in the art will recognize that one or more of the steps of these methods may be omitted or repeated. Still further, the methods may be combined, or conducted simultaneously without departing from the scope of the disclosure.

Although described generally hereinabove, it is envisioned that the memory 32 may include any non-transitory computer-readable storage media for storing data and/or software including instructions that are executable by the processor 30 and which control the operation of the workstation 20 and, in some embodiments, may also control the operation of the medical device 50, and/or imaging device 14. In an embodiment, memory 32 may include one or more storage devices such as solid-state storage devices, e.g., flash memory chips. Alternatively, or in addition to the one or more solid-state storage devices, the memory 32 may include one or more mass storage devices connected to the processor 30 through a mass storage controller (not shown) and a communications bus (not shown).

The description of computer-readable media contained herein refers to solid-state storage, it should be appreciated by those skilled in the art that computer-readable storage media can be any available media that can be accessed by the processor 30. That is, computer readable storage media may include non-transitory, volatile, and non-volatile, removable, and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by the therapy source 24.

### Numbered Clauses

The disclosure can be described in connection with the following numbered clauses.
1. A system for performing a diagnostic procedure, comprising: a medical device configured for placement within a blood vessel and including a light source and an ultrasound transducer disposed on a distal portion of the medical device; and a computing device including a processor and a memory storing instructions, which when executed by the processor, cause the computing device to: cause the light source to deliver a first series of laser pulses to a first location along a wall of a blood vessel; cause the light source to deliver a second series of laser pulses to a second location along a wall of the blood vessel; receive a signal indicative of ultrasonic waves emitted in response to the first series of laser pulses from the ultrasound transducer; receive a second signal indicative of ultrasonic waves emitted in response to the second series of laser pulses from the ultrasound transducer; determine a difference in time between emission of maximum amplitude of ultrasonic waves in response to the first series of laser pulses and emission of a maximum amplitude of ultrasonic waves in response to the second series of laser pulses; and calculate a pulse wave velocity within the blood vessel based on the calculated a distance between the first location and the second location and the determined difference in time, wherein a pulse wave velocity in excess of a threshold is predictive of a response to therapy.
2. The system according to clause 1, wherein the detected ultrasonic waves are generated by hemoglobin present the blood vessel emitted in response to the first or second series of laser pulses.
3. The system according to clauses 1 or 2, wherein the instructions, which when executed by the processor, cause the computing device to: convert the first signal indicative of the detected ultrasonic waves emitted in response to the first series of laser pulses and the second signal indicative of the detected ultrasonic waves emitted in response to the second series of laser pulses to a photoplethysmography signal.
4. The system according to any of clauses 1-3, wherein the first series of laser pulses and the second series of laser pulses have a wavelength of between approximately 200 nm and approximately 1400 nm.
5. The system according to any of clauses 1-4, wherein the first series of laser pulses and the second series of laser pulses have a wavelength of between approximately 600 nm and approximately 700 nm.
6. The system according to any of clauses 1-5, wherein each pulse of the first series of laser pulses has a duration of approximately a picosecond.
7. The system according to any of clauses 1-6, wherein each pulse of the second series of laser pulses has a duration of approximately a nanosecond.
8. The system according to any of clauses 1-7, wherein the light source is a bi-directional laser.
9. The system according to any of clauses 1-8, further comprising a therapeutic assembly disposed on the medical device, the therapeutic assembly configured to denervate nerves of the blood vessel.
10. The system according to clause 9, wherein the therapeutic assembly is configured to deliver monopolar radio-frequency energy.
11. The system according to clause 9, wherein the therapeutic assembly is configured to cool a portion of the blood vessel with a cryoablation medium.
12. A system for performing a diagnostic procedure, comprising: a medical device configured for placement within a blood vessel and including a needle extendible from the medical device and into adventitia of the blood vessel, a light source disposed on a distal portion of the needle, and an ultrasound transducer disposed on a distal portion of the medical device; and a computing device including a processor and a memory storing instructions, which when executed by the processor, cause the computing device to: cause the light source to deliver a series to the adventitia of a blood vessel; detect ultrasonic waves emitted in response to the series of laser pulses; identify an elevated response within a portion of the detected ultrasonic waves compared to the remaining portion of the detected ultrasonic waves, wherein the identified elevated response is indicative of a presence of a nerve fascicle within the blood vessel; and determine a location of the nerve fascicle within the adventitia of the blood vessel.
13. The system according to clause 12, wherein the light source is a microlaser.
14. The system according to clauses 12 or 13, wherein the first series of laser pulses and the second series of laser pulses have a wavelength of between approximately 200 nm and approximately 1400 nm.
15. The system according to any of clauses 12-14, wherein the first series of laser pulses and the second series of laser pulses have a wavelength of between approximately 600 nm and approximately 700 nm.
16. The system according to any of clauses 12-15, further comprising a therapeutic assembly disposed on the medical device, the therapeutic assembly configured to denervate nerves of the blood vessel.
17. The system according to clause 16, wherein the therapeutic assembly is configured to deliver monopolar radio-frequency energy.
18. The system according to clause 16, wherein the therapeutic assembly is configured to cool a portion of the blood vessel with a cryoablation medium.
19. The system according to clause 16, wherein the instructions, when executed by the processor, cause the processor to: cause the light source to deliver a second series of laser pulses to the adventitia of a blood vessel; detect second ultrasonic waves emitted in response to the second series of laser pulses; determine whether an elevated response is within the detected second ultrasonic waves, wherein a lack of the elevated response is indicative of a successful denervation.
20. A method of assessing a target location for therapy, comprising: causing, by a computing device, a light source to deliver a first series of laser pulses to a first location along a wall of a blood vessel; causing, by the computing device, the light source to deliver a second series of laser pulses to a second location along the wall of the blood vessel; receiving, by the computing device, a first signal indicative of detected ultrasonic waves emitted in response to the first series of laser pulses; receiving, by the computing device, a second signal indicative of detected ultrasonic waves emitted in response to the second series of laser pulses; determining, by the computing device, a difference in time between emission of maximum ultrasonic waves in response to the first series of laser pulses and emission of a maximum ultrasonic waves in response to the second series of laser pulses; and calculating, by the computing device, a pulse wave velocity within the blood vessel based on a distance between the first location and the second location and the determined difference in time, wherein a pulse wave velocity in excess of a threshold is predictive of a response to therapy.
21. The method according to clause 20, wherein the detected ultrasonic waves are generated by hemoglobin present the blood vessel in response to the first or second series of laser pulses.
22. The method according to clauses 20 or 21, further comprising converting, by the computing device, the first signal indicative of the detected ultrasonic waves emitted in response to the first series of laser pulses and the second signal indicative of the detected ultrasonic waves emitted in response to the second series of laser pulses to a photoplethysmography signal.
23. The method according to any of clauses 20-22, wherein the first series of laser pulses and the second series of laser pulses have a wavelength of between approximately 200 nm and approximately 1400 nm.
24. The method according to any of clauses 20-23, wherein the first series of laser pulses and the second series of laser pulses have a wavelength of between approximately 600 nm and approximately 700 nm.
25. The method according to any of clauses 20-24, wherein each pulse of the first series of laser pulses has a duration of approximately a picosecond.
26. The method according to any of clauses 20-25, wherein each pulse of the second series of laser pulses has a duration of approximately a nanosecond.
27. The method according to any of clauses 20-26, wherein the blood vessel is one or more of a renal artery, a hepatic artery, a splanchnic artery, or a mesenteric artery.
28. The method according to any of clauses 20-27, further comprising causing, by the computing device, a therapy source to generate a signal for the denervation of sympathetic nerves enervating the blood vessel.
29. The method according to clause 28, further comprising: causing, by a computing device, a light source to deliver a third series of laser pulses to the first location along a wall of a blood vessel; causing, by the computing device, a light source to deliver a fourth series of laser pulses to the second location along a wall of the blood vessel; receiving, by the computing device, a third signal indicative of detected ultrasonic waves emitted in response to the third series of laser pulses; receiving, by the computing device, a fourth signal indicative of detected ultrasonic waves emitted in response to the fourth series of laser pulses; determining, by the computing device, a second difference in time, the second difference intime being between emission of a maximum amplitude of ultrasonic waves in response to the third series of laser pulses and emission of a maximum amplitude of ultrasonic waves in response to the fourth series of laser pulses; and calculating, by the computing device, a pulse wave velocity within the blood vessel based on a calculated distance between the first and second locations and the determined second difference in time, wherein a pulse wave velocity less that a predetermined threshold is indicative of a successful denervation of the blood vessel.
30. The method according to any of clauses 20-29, further comprising determining, by the computing device, a distance of the light source from the wall of the blood vessel.
31. The method according to any of clauses 20-30, wherein the first series of pulses are emitted from the light source at an angle relative to the second series of pulses.
32. The method according to any of clauses 20-31, further comprising determining, by the computing device, the distance between the first location the second location based on the angle and the distance of the light source from the wall of the blood vessel.
33. The method according to any of clauses 20-32, further comprising converting, by the computing device, the signal indicative of the detected ultrasonic waves emitted in response to the first series of laser pulses, the second series of laser pulses, the third series of laser pulses or the fourth series of laser pulses to a photoacoustic signal.
34. A method for performing a diagnostic procedure, comprising: causing, by a computing device, a light source to deliver a first series of laser pulses to an adventitia of a blood vessel; receiving, by the computing device a signal indicative of detected ultrasonic waves emitted in response to the first series of laser pulses; identifying, by the computing device, an elevated response within a portion of the detected ultrasonic waves compared to a remaining portion of the detected ultrasonic waves, wherein the identified elevated response is indicative of a presence of a nerve fascicle within the adventitia of the blood vessel; and determining, by the computing device, a location of the nerve fascicle based on the identified elevated response.
35. The method according to clause 34, further comprising correlating, by the computing device, the identified elevated response to a calcium channel disposed within a nerve fascicle.
36. The method according to clauses 34 or 35, wherein the light source is operably connected to a distal end portion of a needle configured to extend from medical device in the blood vessel into adventitia of the blood vessel.
37. The method according to any of clauses 34-36, wherein the first series of laser pulses have a wavelength of between approximately 200 and 1400 nm.
38. The method according to any of clauses 34-37, wherein the first series of laser pulses have a wavelength of between approximately 600 nm and 700 nm.
39. The method according to any of clauses 34-38, wherein the blood vessel is one or more of a renal artery, a hepatic artery, a splanchnic artery, or a mesenteric artery.
40. The method according to any of clauses 34-39, further comprising causing, by the computing device, a therapy source to generate a signal for the denervation of sympathetic nerves enervating the blood vessel.
41. The method according to clause 40, further comprising: causing, by the computing device, the light source to deliver a second series of laser pulses to the adventitia of a blood vessel; receiving, by the computing device, a signal indicative of detected ultrasonic waves emitted in response to the second series of laser pulses; determining, by the computing device, whether an elevated response is within the detected second ultrasonic waves, wherein a lack of the elevated response is indicative of a successful denervation of the blood vessel.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of embodiments. Those skilled in the art will envision other modifications within the scope and spirit of the claims appended hereto.

1. A system for performing a diagnostic procedure, comprising:
a medical device configured for placement within a blood vessel and including a light source and an ultrasound transducer disposed on a distal portion of the medical device; and
a computing device including a processor and a memory storing instructions, which when executed by the processor, cause the computing device to:
cause the light source to deliver a first series of laser pulses to a first location along a wall of a blood vessel;
cause the light source to deliver a second series of laser pulses to a second location along a wall of the blood vessel;
receive a signal indicative of ultrasonic waves emitted in response to the first series of laser pulses from the ultrasound transducer;
receive a second signal indicative of ultrasonic waves emitted in response to the second series of laser pulses from the ultrasound transducer;
determine a difference in time between emission of maximum amplitude of ultrasonic waves in response to the first series of laser pulses and emission of a maximum amplitude of ultrasonic waves in response to the second series of laser pulses; and
calculate a pulse wave velocity within the blood vessel based on the calculated a distance between the first location and the second location and the determined difference in time,
wherein a pulse wave velocity in excess of a threshold is predictive of a response to therapy.

2. The system according to claim 1, wherein the ultrasonic waves are generated by hemoglobin present the blood vessel emitted in response to the first or second series of laser pulses.

3. The system according to claim 1, wherein the instructions, which when executed by the processor, cause the computing device to:
convert the first signal indicative of the ultrasonic waves emitted in response to the first series of laser pulses and the second signal indicative of the ultrasonic waves emitted in response to the second series of laser pulses to a photoplethysmography signal.

4. The system according to claim 1, wherein the first series of laser pulses and the second series of laser pulses have a wavelength of between approximately 200 nm and approximately 1400 nm.

5. The system according to claim 4, wherein the first series of laser pulses and the second series of laser pulses have a wavelength of between approximately 600 nm and approximately 700 nm.

6. The system according to claim 1, wherein each pulse of the first series of laser pulses has a duration of approximately a picosecond or approximately a nanosecond.

7. The system according to claim 1, wherein the light source is a bi-directional laser.

8. The system according to claim 1, further comprising a therapeutic assembly disposed on the medical device, the therapeutic assembly configured to denervate nerves of the blood vessel.

9. The system according to claim 8, wherein the therapeutic assembly is configured to deliver monopolar radio-frequency energy or is configured to cool a portion of the blood vessel with a cryoablation medium.

10. A system for performing a diagnostic procedure, comprising:
a medical device configured for placement within a blood vessel and including a needle extendible from the medical device and into adventitia of the blood vessel, a light source disposed on a distal portion of the needle, and an ultrasound transducer disposed on a distal portion of the medical device; and
a computing device including a processor and a memory storing instructions, which when executed by the processor, cause the computing device to:
cause the light source to deliver a series of laser pulses to the adventitia of a blood vessel;
detect photoacoustic signals emitted in response to the series of laser pulses;
identify an elevated response within a portion of the detected photoacoustic signals compared to the remaining portion of the detected photoacoustic signals, wherein the identified elevated response is indicative of a presence of a nerve fascicle within the adventitia of the blood vessel; and
determine a location of the nerve fascicle within the adventitia of the blood vessel.

11. The system according to claim 10, wherein the light source is a microlaser.

12. The system according to claim 11, wherein the series of laser pulses have a wavelength of between approximately 200 nm and approximately 1400 nm.

13. The system according to claim 12, wherein the series of laser pulses have a wavelength of between approximately 600 nm and approximately 700 nm.

14. The system according to claim 10 further comprising a therapeutic assembly disposed on the medical device, the therapeutic assembly configured to denervate nerves of the blood vessel.

15. The system according to any claim 10 wherein the instructions, when executed by the processor, cause the processor to:
cause the light source to deliver a second series of laser pulses to the adventitia of a blood vessel;
detect second photoacoustic signals emitted in response to the second series of laser pulses; and
determine whether an elevated response is within the detected second photoacoustic signals, wherein a lack of the elevated response is indicative of a successful denervation.

16. A method of assessing a target location for therapy, comprising:
causing, by a computing device, a light source to deliver a first series of laser pulses to a first location along a wall of a blood vessel;
causing, by the computing device, the light source to deliver a second series of laser pulses to a second location along the wall of the blood vessel;
receiving, by the computing device, a first signal indicative of detected ultrasonic waves emitted in response to the first series of laser pulses;
receiving, by the computing device, a second signal indicative of detected ultrasonic waves emitted in response to the second series of laser pulses;
determining, by the computing device, a difference in time between emission of maximum ultrasonic waves in response to the first series of laser pulses and emission of a maximum ultrasonic waves in response to the second series of laser pulses; and
calculating, by the computing device, a pulse wave velocity within the blood vessel based on a distance between the first location and the second location and the determined difference in time, wherein a pulse wave velocity in excess of a threshold is predictive of a response to therapy.

17. The method according to claim 16, wherein the detected ultrasonic waves are generated by hemoglobin present the blood vessel in response to the first or second series of laser pulses.

18. The method according to claim 16, further comprising converting, by the computing device, the first signal indicative of the detected ultrasonic waves emitted in response to the first series of laser pulses and the second signal indicative of the detected ultrasonic waves emitted in response to the second series of laser pulses to a photoplethysmography signal.

19. The method according to claim 16, wherein the first series of laser pulses and the second series of laser pulses have a wavelength of between approximately 200 nm and approximately 1400 nm.

20. The method according to any of claim 16, wherein the first series of laser pulses and the second series of laser pulses have a wavelength of between approximately 600 nm and approximately 700 nm.
